(19) United States
(12) Patent Application Publication
(10) Pub. No.: US 2024/0368526 A1
(43) Pub. Date: Nov. 7, 2024

(54) METHODS FOR INCREASING ALGAE PRODUCTIVITY

(71) Applicant: Global Algae Technology, LLC, San Diego, CA (US)

(72) Inventors: David A. Hazlebeck, San Diego, CA (US); Rodney Corpuz, Lihue, HI (US); Agnieszka Pinowska, San Diego, CA (US); Jesse Traller, Santee, CA (US); Tyson Burch, Golden, CO (US); Matthew Posewitz, Golden, CO (US)

(21) Appl. No.: 18/652,580

(22) Filed: May 1, 2024

Related U.S. Application Data
(60) Provisional application No. 63/463,223, filed on May 1, 2023.

(51) Int. Cl. C12N 1/12

(52) U.S. Cl. CPC C12N 1/12

(57) ABSTRACT

The present disclosure describes cultivation methods for increasing productivity of algae strains. The methods include diluting the algae cultures at specific times, as well as nutrient feeding the cultures at specific times.

### CROSS-REFERENCE TO RELATED APPLICATIONS

This application claims the benefit of and priority to U.S. Provisional Application No. 63/463,223, filed on May 1, 2023, the content of which is incorporated by reference in its entirety.

### STATEMENT REGARDING FEDERALLY SPONSORED RESEARCH

This invention was made with government support under Grant No. DE-EE0008903, and Grant No. DE-EE0008245 awarded by the U.S. Department of Energy. The government has certain rights in the invention.

### BACKGROUND OF THE INVENTION

Algae cultivation has become widely recognized as a promising source of food, feed, biofuel, chemicals, polymers and nutraceuticals. Photosynthetic algae production offers the potential for an order of magnitude higher agricultural productivity than other plants. Some algae strains are preferred because of higher lipid content, better lipid profiles, or higher value compounds. Examples of some strains of commercial interest are Nitzschia for lipid content and profile; Haematococcus for astaxanthin content; Nannochloropsis and a variety of diatoms for EPA content; and Spirulina for phycocyanin and protein content.

As a biofuel source, for example, algae is known to be one of the most efficient phototrophs for converting solar energy into cell growth. Importantly, the use of algae as a biofuel source presents no exceptional problems, i.e., biofuel can be processed from algae as easily as from land-based plants. Also, algae can be grown heterotrophically or mixotrophically to produce materials for biofuel production. For example, microalgae can be grown on cellulosic and hemicellulosic sugars to produce lipids.

Processes involved in creating biofuel from plants are expensive relative to the process of extracting and refining petroleum. It is possible, however, that the cost of processing a plant-derived biofuel could be reduced by maximizing the rate of growth of the plant source. While algae can efficiently transform solar energy into chemical energy via a high rate of cell growth, it has been difficult to create environments in which cell growth rates are optimized. Therefore, the economics of large-scale cultivation remains challenging. One way to improve the economics is through obtaining higher productivity in the cultivation systems, so numerous approaches have been taken to improve the productivity of various cultivation systems.

In light of the above, it is an object of the present invention to provide a method for maximizing the growth of algae cultures. During algae cultivation, nutrients are typically added to the cultures all at once and often during daylight hours or in the middle of the day. Additionally, algae dilution and transfer occurs in the middle of the day, and often simultaneously with nutrient feeding. While this may be cost beneficial to hours of operation, increased algae productivity may be achieved by altering the timing of culture dilution and/or nutrient delivery to periods when the algae is not receiving optimal light.

### SUMMARY OF THE INVENTION

The disclosure provides novel cultivation methods for increasing the productivity of algae strains.

In one aspect of the disclosure, provided herein is a method for cultivating an algae strain in an aqueous culture, the method comprising adding a diluent to the culture daily at a time period selected from: a) no earlier than one hour before sunset and no later than two hours before sunrise; and b) no earlier than two hours before sunrise and no later than one hour before sunset, wherein the diluent is added gradually.

In embodiments, the diluent is added at time period a); and the entire diluent is added to the culture at one time. In embodiments, the diluent is added at time period a); and before adding the diluent to the culture each day, the method further comprises transferring the culture from a first container to a second container, wherein the second container has a greater area than the first container. In embodiments, the diluent is added at time period b); and before adding the diluent to the culture each day, the method further comprising transferring the culture from a first container to a second container, wherein the second container has a greater area than the first container. In embodiments, the diluent comprises one or more of aqueous media, water, and recycled media from an algae harvest.

In another aspect, provided herein is a method for cultivating an algae strain in an aqueous culture, the method comprising: a) adding a first subset of nutrients to the culture daily no earlier than one hour before sunset and no later than two hours before sunrise; b) adding a second subset of nutrients to the culture daily no earlier than 2 hours before sunrise and ending no later than sunset, wherein the one or more nutrients are added gradually; and c) adding a third subset of nutrients to the culture daily no earlier than two hours before sunrise and no later than two hours after sunrise.

In embodiments, the first subset of nutrients comprises one or more of a phosphorous source, a calcium source, a magnesium source, a sulfur source, and an iron source.

In embodiments, the first subset of nutrients and the second subset of nutrients each comprise one or more of a nitrogen source, a calcium source, a magnesium source, and a potassium source; and the third subset of nutrients comprises a phosphorus source.

In embodiments, the first subset of nutrients comprises one or more of a calcium source, a magnesium source, and a potassium source; the second subset of nutrients comprises a nitrogen source; and the third subset of nutrients comprises one or more of a calcium source, a magnesium source, and a potassium source.

In embodiments, the third subset of nutrients further comprises a nitrogen source.

In embodiments, the method further comprising adding a fourth subset of nutrients to the culture daily such that the concentration of the fourth subset of nutrients in the media is kept substantially constant throughout the day.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 is a schematic of an algae cultivation system for performing the methods described herein.

FIG. 2 is a schematic of a second algae cultivation system for performing the methods described herein.

FIGS. 3A-3C depict mean productivity in g AFDW m⁻² d⁻¹ for GAI-337 under three dilution time regimes (dawn, midday, and dusk) calculated using the correlation between AFDW and OD₇₅₀ (A) and using the actual AFDW filter protocol (B). (C) compares ash content as a percentage of dry biomass for the three dilution time regimes. Cultures were grown under high oxygen stress diel-cycle conditions over four consecutive days in biological duplicate for each dilution time regime (n=8 for each regime). Error bars represent ±1 standard deviation. Statistical comparison of means was done by t-test (two-tailed, two-sample homoscedastic, p≤0.05=*, p≤0.01=**, p≤0.001=***).

FIG. 4 depicts correlations between AFDW (g L⁻¹) and OD₇₅₀ for sampling and dilution at dawn, midday, and dusk. These correlations were used to improve estimates of areal biomass productivities shown in FIG. 3A.

FIG. 5 depicts average growth phase productivity for Nitzschia inconspicua GAI-370. Triplicate 1.8 m² outdoor raceways were fed nutrients and diluted at within 2 hours of sunset (night feed, blue bars), within 3 hours of sunrise (morning feed, yellow bars), or diluted within 2 hours of sunset and fed nutrients within 1 hour of sunrise (dilute night, feed morning, green bar). For the latter, only a single replicate was run. Productivities are based upon the daily ash-free dry weight biomass (AFDW in grams) per meter squared per day. KAF-XXXX denotes the batch ID given for each treatment. Daily productivity is averaged across the growth phase portion of the experiment, which operated for 21 days from Aug. 12, 2022-9/1/22.

FIG. 6 depicts the ash percentage of the samples shown in FIG. 5.

FIG. 7 depicts average growth phase productivity for GAI-370. Single outdoor raceways were fed nutrients and diluted at dusk (blue line), fed nutrients and diluted in the morning (yellow line), and fed nutrients in the morning and metered diluted at night for 5 hours (green line).

FIG. 8 depicts the impact of nutrient feed timing of the feeding on productivity during winter time and with algae cultures under direct air capture conditions.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

Provided herein are cultivation methods for increasing productivity of algae strains.

### Dilution Timing

In an aspect, a method for cultivating an algae strain in an aqueous culture is provided, the method comprising diluting the culture every day in the evening. In an embodiment, the culture is diluted at a time when the sunlight is producing no more than 300 microeinsteins per square meter per second of photosynthetically active radiation. In an embodiment, the culture is diluted between no earlier than one hour before sunset and no later than two hours before sunrise. Or the culture may be diluted via metered dilution at a rate of less than 1% change in volume of the culture per two minutes, three minutes, five minutes, or ten minutes.

An “algae culture” is an algae growth or “slurry” in an aqueous environment such as water or a medium. The algae culture is grown in a container that retains liquid, such as a pond, a tank, or a raceway.

As used herein, “dilution” refers to the process of adding a diluent to the algae culture, wherein a culture is considered “diluted” when the concentration of algae in the culture is reduced by at least about 10%. The diluent may be aqueous media, water, or both. The aqueous media may be recycled media from a previous harvest, i.e., media remaining in a harvest after the algae slurry has been removed.

In another aspect, a method for cultivating an algae strain in an aqueous culture is provided, the method comprising gradually diluting the medium throughout the day. For gradual daytime dilution, the culture may be diluted no earlier than two hours before sunrise and no later than one hour before sunset. The gradual dilution may be performed by adding diluent over a period of time. The diluent may be added at rate that results in less than 1% change in volume of the culture per two minutes, three minutes, five minutes, or ten minutes.

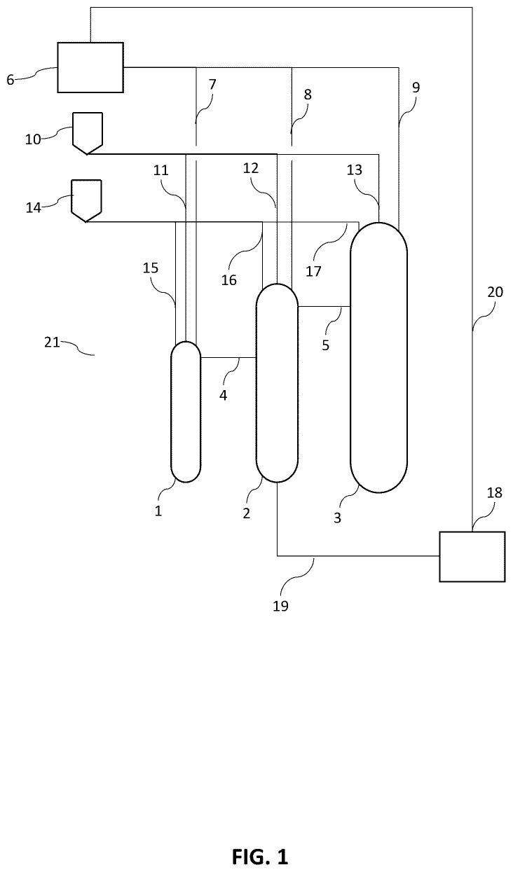

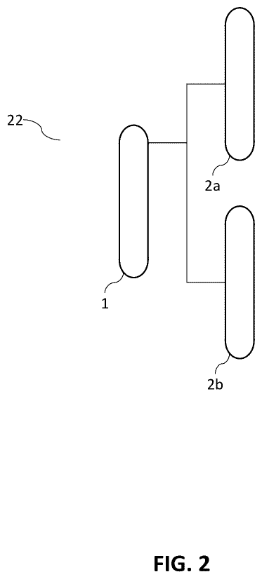

The dilution may be performed using an algae cultivation system. In some embodiments, the dilution is performed in an algae cultivation system as described in FIG. 1 or FIG. 2.

The system 21 of FIG. 1 includes: a first raceway 1, a second raceway 2 having a greater surface area than the first raceway, a third raceway 3 having a greater area than the second raceway, a conduit or aqueduct 4 to transfer algae slurry from the first raceway to the second raceway, a conduit or aqueduct 5 to transfer algae slurry from the second raceway to the third raceway, a media tank or pond 6, a conduit or aqueduct 7 to transfer media to the first raceway from the media tank or pond, a conduit or aqueduct 8 to transfer media to the second raceway from the media tank or pond, a conduit or aqueduct 9 to transfer media to the third raceway from the media tank or pond, a first nutrient tank 10, a conduit 11 to transfer nutrients from the first nutrient tank to the first raceway, a conduit 12 to transfer nutrients from the first nutrient tank to the second raceway, a conduit 13 to transfer nutrients from the first nutrient tank to the third raceway, a second nutrient tank 14, a conduit or aqueduct 15 to transfer nutrients from the second nutrient tank to the first raceway, a conduit or aqueduct 16 to transfer nutrients from the second nutrient tank to the second raceway, a conduit or aqueduct 17 to transfer nutrients from the second nutrient tank to the third raceway, an algae harvest system 18, a conduit or aqueduct 19 to transfer algae slurry to the harvest system, and a conduit or aqueduct 20 to recycle media from the harvest system to the media tank or pond. A harvest system is a system configured to produce a concentrated algae slurry product and a recycled media from the slurry. Examples of suitable harvest systems are described in U.S. Pat. No. 10,501,721 B2, U.S. Pat. No. 10,351,815 B2, and U.S. Patent Publication No. US2020/0299636A1, each of which are incorporated by reference, in their entirety and for all purposes, herein.

FIG. 2 illustrates a second algae cultivation system 22, having an alternate configuration than system 21. In addition to elements 4-20 (not shown), system 22 includes: a first raceway 1, a second raceway 2a, and a third raceway 2b. In this system 22, instead of being transferred from a smaller raceway, the algae slurry is transferred from a raceway 1 into two raceways 2a and 2b such that the combined area of the two or more raceways is greater than the smaller raceway.

Systems 21 and 22 may be operated in at least two different ways; a conventional mode or a single pass batch reactor (SPBR) mode. In conventional mode, a portion of the algae slurry is transferred from raceway 2 of system 21 (or 2a and 2b of system 22) to the harvest system 18 or to another raceway (e.g. third raceway 3). Media is added from the media tank 6 to the raceway 2 (or 2a and 2b) to replace at least a portion of the harvested volume to facilitate additional growth.

In SPBR mode, substantially all of the culture is pumped out of a raceway (e.g. second raceway 2) into another raceway 3 or the harvest system 18 each day, every two days, or every three days. A new algae solution is transferred from a smaller raceway 1 into the second raceway 2 (or 2a and 2b). Then, media is added from the media tank 6 to the raceway 2 (or 2a and 2b), which dilutes the culture to facilitate additional growth.

In both conventional mode and SPBR mode, media is added to increase the liquid depth in the raceway 2 (or 2a and 2b) and reduce the algae concentration to facilitate additional growth. The media tank or pond 6 can include a diluent of water or recycled media from a harvest, or a combination of the two.

### Nutrient Addition Timing

During algae cultivation, nutrients are typically added to the cultures one time in the morning. The inventors have discovered that productivity is increased by adding nutrients at night. Therefore, in another aspect, a method for cultivating an algae strain in an aqueous culture is provided, the method comprising adding nutrients to the culture in the evening. In an embodiment, the nutrients are added at a time when the sunlight is producing no more than 300 microeinsteins per square meter per second of photosynthetically active radiation. In an embodiment, the nutrients are added at no earlier than one hour before sunset and no later than two hours before sunrise. The nutrients may be added using system 21 or 22, wherein the nutrients may be provided from first nutrient tank 10 and/or second nutrient tank 14. Each nutrient tank may comprise at least one of nitrate, urea, ammonium ion, silicate, phosphate, potassium, calcium, magnesium, iron and sulfate. The nitrate may be added to the nutrient tank in the form of nitric acid, sodium nitrate, potassium nitrate, or ammonium nitrate. The ammonium ion may be added to the nutrient tank in the form ammonia, ammonium nitrate, mono ammonium phosphate, diammonium phosphate, ammonium chloride, ammonium bicarbonate, or ammonium carbonate. The silicate may be added to the nutrient tank in the form of sodium silicate wherein the ratio of silica to disodium oxide varies from 1 to 3.4. The phosphate may be added to the nutrient tank in the form of mono, di, or tri sodium phosphate; mono, di, or tri potassium phosphate; or mono or di ammonium phosphate. The potassium may be added to the nutrient tank in the form of potassium nitrate, potassium chloride, potassium carbonate, potassium bicarbonate, potassium sulfate, mono potassium phosphate, di potassium phosphate, tri potassium phosphate, or potassium hydroxide. The calcium may be added to the nutrient tank in the form of calcium chloride. The magnesium may be added to the nutrient tank in the form of magnesium sulfate or magnesium chloride. The iron may be added to the nutrient tank in the form of ferric chloride, iron complexed with EDTA, or iron complexed with citric acid. Once the nutrients are mixed, they are in the form of ions in solution, regardless of the original form in which they are added. Alternative compounds may be used in addition to those described above depending on availability, cost, desire for organic algae, or other preferences.

All nutrients may or may not be added at the same time. For example, in an embodiment, a nutrient or group of nutrients are added in the morning, i.e., between two hours before sunrise and two hours after sunrise. Another nutrient or group of nutrients are added gradually throughout the day and/or night, and are optionally controlled to maintain a minimum concentration of the nutrient in solution in the culture. Another nutrient or group of nutrients are at mid-day, i.e. within two hours of the midpoint between sunrise and sunset. Another nutrient or group of nutrients are added at night, i.e. between one hour before sunset and two hours before sunrise.

Accordingly, the method may comprise a) adding a first subset of nutrients to the culture daily no earlier than one hour before sunset and no later than two hours before sunrise, e.g. at night; b) adding a second subset of nutrients to the culture daily no earlier than 2 hours before sunrise and ending no later than sunset, wherein the one or more nutrients are added gradually, e.g. throughout the day; and c) adding a third subset of nutrients to the culture daily no earlier than two hours before sunrise and no later than two hours after sunrise, e.g. in the morning.

Some nutrients are believed to be consumed luxuriously by algae, meaning the algae will take up more of a nutrient than needed for functioning and production when nutrient levels are above optimum. Luxuriously consumed nutrients, depending on their impact on algae growth and survival, may not be beneficial when provided in excess. For example, phosphorous, calcium, magnesium, sulfur, and some micronutrients, such as iron, are believed to be consumed luxuriously. These nutrients may be added at night to avoid negatively impacting the equilibrium of the culture. In this embodiment, the first subset of nutrients comprises one or more of phosphorous, calcium, magnesium, sulfur, and iron. The first subset of nutrients may comprise additional nutrients, and some of the nutrients enumerated may be added to the culture at times other than at night.

In an exemplary embodiment, a phosphorous source (e.g. phosphate) is added in the morning; and one or more of a nitrogen source (e.g. nitrate, urea, ammonia), a calcium source, a magnesium source, and a potassium source are added at night or gradually throughout the day. In this embodiment, the first subset and/or the second subset of nutrients comprise one or more of a nitrogen source (e.g. nitrate, urea, ammonia), a calcium source, a magnesium source, and a potassium source; and the third subset of nutrients comprises phosphorous. Each subset of nutrients may comprise additional nutrients, and some of the nutrients enumerated may be added to the culture at other times.

In another embodiment, a phosphorous source is added in the morning; a calcium source, a magnesium source, and a potassium source are added at night; and a nitrogen source is added gradually throughout the day and/or night, and kept substantially constant at a minimum level. In this embodiment, the first subset of nutrients comprises a calcium source, a magnesium source, and a potassium source; the second set of nutrients comprises a nitrogen source; and the third subset of nutrients comprises a calcium source, a magnesium source, a potassium source, and optionally a nitrogen source. Each subset of nutrients is not exclusive to the enumerated nutrients, and some nutrients enumerated in a subset may be added to the culture at other times.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1. Assessment of Dilution Timing on Areal Biomass Productivity

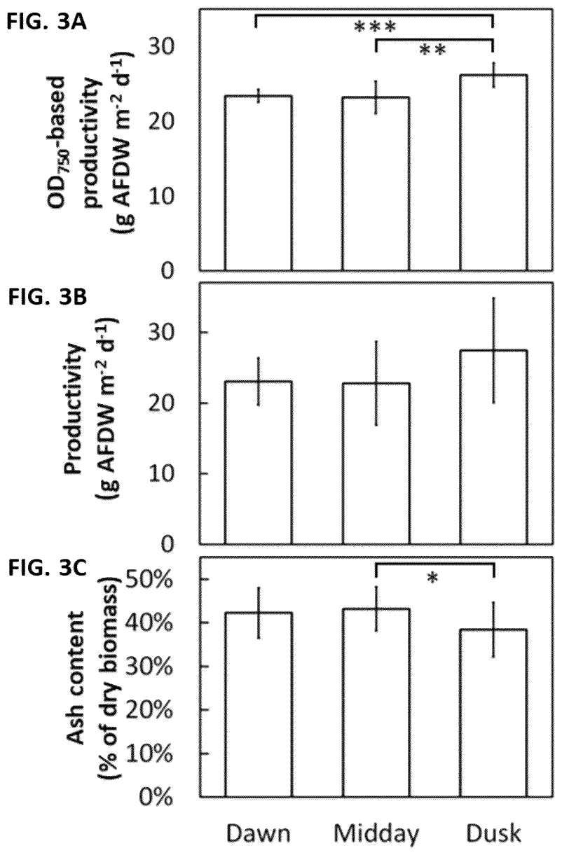

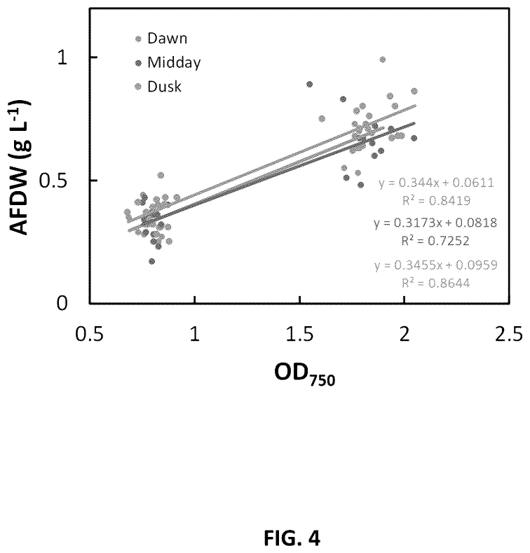

It was previously determined that high oxygen tolerant Nitzschia inconspicua strain GAI-337, had improved biomass productivities under high O₂ stress. To assess the optimal culture management strategies to maximize biomass bioproductivities, different sampling and dilution times on productivity and ash content were tested. Cultures were grown under high O₂ stress conditions (supersaturation), sampled and diluted just before sunrise (dawn), at noon during the day (midday), or just after sunset (dusk). It was hypothesized that diluting during the day might cause a shock to the cultures by taking them from a dense state to a more dilute state. In the dilutes state the cells receive more light on average, the nutrient concentration is suddenly reduced, and the cellular concentration is suddenly reduced. The sudden change might stress the cells because the photosynthetic apparatus, intercellular sensing, and nutrient uptake systems would be tuned for the dense state. It was also hypothesized that cultures might shift from growth to nutrient scavenging if new nutrients were made available during the day since diatoms are reported to hoard nutrients to gain a competitive advantage in aquatic ecosystems (Hildebrand et al. 2012). It was found that there was no significant difference between pre-sunrise dilution and midday (FIGS. 3A and 3B), suggesting that the reduced growth rate from dilution persists for at least several hours. The stress may lower growth rates in a similar way, sending cells into a lag-phase in heterotrophic culture when conditions are suddenly changed. However, diluting just after sunset led to higher biomass productivity when using OD₇₅₀-based productivity estimates (FIG. 3A), which was likely due to the cells bearing more biomass at the end of the growing day compared to sunrise (after respiring at night) and midday. This hypothesis was supported by the lower ash percentage and higher AFDW of dusk dilution time samples (FIG. 3C). OD₇₅₀-based productivity estimates were included to compare productivities because high variability was observed in AFDW measurements. FIG. 4 shows the correlation curves for AFDW vs OD₇₅₀ for the three sampling times used to generate the OD₇₅₀-based productivity estimates. While in the laboratory, invaders such as bacteria, fungi, and other algae are reduced. Therefore, it was not clear whether a dusk dilution and feeding strategy would be viable in outdoor open ponds because nutrients would be made available when sunlight would not be, which might increase nutrient availability to contaminating organisms. Therefore, as discussed below, these results were tested in outdoor ponds to assess the effect of dusk dilution and feeding on areal productivities.

### Methods for Example 1

### Strain Isolation and Identification

Nitzschia inconspicua str. hildebrandi GAI-229 was collected from the tidal area of a stream on the island of Kauai, Hawaii, USA. The strain was identified as a diatom from the genus Nitzschia by physiological and genetic characteristics (see Oliver et al. 2021).

### Generation of High-Oxygen Strain GAI-337

The wildtype strain Nitzschia inconspicua str. hildebrandi GAI-229 was placed into a turbidostat photobioreactor under constant temperature (24° C.), pH >9 and light at a greater intensity than the saturating intensity for the strain. The O₂ concentration in the sparging gas was gradually increased, leading to high O₂ selective pressure while simultaneously selecting for culture growth (slower growing cells were quickly diluted out of the culture). As the O₂ concentration approached ˜80% of the sparging gas (˜380% air saturation as measured in the liquid), growth was completely inhibited. At that point, the O₂ concentration in the sparging gas was decreased and the culture was allowed to recover for 12.5 days. This procedure was repeated 3 consecutive times and the improvement in growth was monitored in relation to the O₂ concentration. The high O₂ adaptation appeared within the strain during the first round of this process, and additional rounds of attempted directed evolution afterward did not appear to lead to additional improvement.

### Strain Cultivation and Maintenance

### Culture Medium

Strains were grown in brackish media as described previously (Quigg and genome papers). All culture medium was filter sterilized using Nalgene Rapid-Flow bottle top vacuum filters (Nalgene, USA).

### Culture Growth and Maintenance Prior to Experimentation

20 mL cultures were initially grown in 50 mL flasks and scaled up to 400 mL cultures in 1 L flasks prior to inoculation in flat-sided bottles. Flask cultures were grown at room temperature (˜23° C.) on orbital shaker tables (110 RPM) with constant light (100 μmol photons m⁻² s⁻¹). Cultures were regularly diluted as necessary to maintain growth in the flasks without nutrient or light limitation.

After a suitable amount of biomass was accumulated, 400 mL cultures were transferred to pond-mimicking conditions in 500 mL flat-sided bottles (No. 1396, Pyrex, Germany) with stainless steel bubble tubes extending 2 cm below the culture surface. Inlet and outlet gas was filter-sterilized by Acro 37 TF vent devices (Pall Corporation, USA). Inlet gas was provided by aquarium pumps or by mass flow controllers. Cultures were illuminated by cool white LEDs with water-cooled heatsinks attached to the back of the LED panels. The diel light regime was programed to mimic a solar day in Kauai in mid-June (29.8 MJ m⁻² d⁻¹) with the light intensity measured by an integrating sphere in the middle of a water-filled experimental bottle. The maximum light intensity during the light cycle was 2,200 μmol photons m² s⁻¹ as measured by the spherical probe, which corresponded to 2,300 μmol photons m² s⁻¹ as measured by a flat probe placed against the glass inside the center of an empty experimental bottle. 50 mm computer fans were positioned to blow cooling air between the LEDs and the culture bottles. Culture temperatures were either allowed to fluctuate naturally, being heated by the lights and cooled by the ambient air, which mimicked natural temperature fluctuations seen in outdoor ponds in June in Kauai, or controlled by a programmable water bath connected to a plexiglass enclosure housing the culture bottles. After transfer to pond-mimicking flat-sided bottles, cultures were allowed to acclimate for three to seven days prior to the start of an experiment.

### Strain Storage and Revival

Strains were grown in shaker flasks as above, then frozen by the methods of Elliot et al. (2012) at −80° C. Briefly, 1.9 mL of culture was transferred to a 2 mL Nalgene cryogenic tube in a sterile hood. 100 μL of DMSO (brand) was added, the tube was closed and gently inverted several times and quickly placed into a pre-chilled (4° C.) Mr. Frosty (Nalgene, USA) which was then quickly placed into a −80° C. freezer. The tubes were kept at −80° C. until revival. For revival, the tube was held in a water bath at 37° C., inverted occasionally until no ice remained, then centrifuged for 1 minute at 1000 RPM. The supernatant was quickly aspirated and the cells were resuspended in fresh culture medium. After a second round of centrifugation and aspiration of the supernatant as above, the cells were resuspended in fresh culture medium and transferred to a 50 mL shaker flask with a total medium volume of 15 mL. The flask was kept in low light on a benchtop for 1 day, then covered with a Kimwipe® to provide shade, and placed on an orbital shaker table. After the culture showed signs of growth (1-2 weeks depending on the strain and frozen culture density), the shade was removed. After several days of growth, the entire culture was transferred to a 250 mL flask and the volume was set to 100 mL with fresh nutrient enriched culture medium.

### Experimental Diel-Cycle Pond-Mimicking Culture Conditions

Triplicate 400 mL cultures were grown in 500 mL flat-sided bottles as described above with temperature control provided by a plexiglass water bath connected to a programmable water bath set to mimic the diel temperature swings seen in outdoor pond cultures in Kauai. For different maximum temperature experiments, the temperature curve was adjusted higher or lower with the minimum temperature (24° C.) remaining the same. Cultures were bubbled with house air regulated by a mass flow controller set to deliver the required air-flow rates as indicated. Cultures were sampled in situ through a side port comprised of a tube clamp and one-way check valve connected to the bubble tube. To sample, the gas line above the sample port was clamped, culture was drawn through the bubble tube and sample port to flush the line with culture and discarded, then the sample was extracted. Due to the rapid settling rate of this diatom strain, in situ sampling was employed as the best method for consistent sampling. Cultures were sampled and diluted with fresh medium once per day just after sunset (dusk). Cultures were diluted to 1.25 OD₇₅₀ (˜0.45 g L⁻¹ AFDW) unless otherwise indicated.

### High Oxygen Stress Diel-Cycle Conditions

Cultures grown to maximize oxygen stress were cultured as above, but without bubbling during the day to maximize the amount of photosynthetically-produced oxygen in the bottles. The cultures were bubbled at night with air (100 mL min⁻¹ bottle⁻¹) to prevent anaerobic culture conditions. The OD₇₅₀ after dilution for these experiments was set to 0.8. Temperatures were allowed to vary naturally as described above with a low temperature at night around 23° C. and a maximum temperature during the day around 33° C.

### Dilution Time Experiments

Cultures were run under high oxygen stress diel-cycle conditions described above and sampled and diluted with fresh medium just before sunrise (dawn, 6 am), at midday (12 pm, maximum light intensity), or just after sunset (dusk, 7 pm). The OD₇₅₀ setpoint for these experiments was 0.8 after dilution.

### OD₇₅₀

One mL of culture was rigorously pipetted to break apart any clumps within the sample, then 100 mL was mixed with 900 mL fresh culture medium in a cuvette and again pipetted rigorously. The optical density at 750 nm (OD₇₅₀) was measured using a DU800 UV-visible spectrophotometer (Beckman Coulter, USA) blanked with fresh culture medium. The OD₇₅₀ was read three times for each sample, pipetting the sample quickly just prior to each measurement, and the mean of the three measurements was recorded. This method was necessary to improve the accuracy of the OD₇₅₀ measurements because the cells settled rapidly and exhibited variability in light scattering for each scan.

### AFDW

10 mL of culture was transferred to a 15 mL falcon tube and centrifuged at 3000 RPM for 5 minutes at room temperature. The supernatant was discarded, and the pellet resuspended in 1 mL of 0.5 M ammonium formate by gently pipetting 6-8 times. 10 additional mL of ammonium formate was added and the 1 mL pipette was rinsed in the cell suspension to remove as many of the cells as possible. The tube was centrifuged again as above and the supernatant was discarded. The cell pellet was again suspended in 1 mL of 0.5 M ammonium formate and vacuum filtered onto a pre-ashed (550° C. for 30 minutes) GF/F glass fiber filter (Whatman, United Kingdom) loaded into a vacuum filtration unit connected to a vacuum pump. The remaining cells were rinsed from the falcon tube with 1 mL 0.5 M ammonium formate and applied to the filter. The filters were placed into aluminum trays and dried at 105° C. overnight. The dry filters were weighed to yield dry weights, then ashed at 550° C. for 2 hours. The ash free filters were then placed into a 105° C. oven overnight, and quickly removed and weighed. AFDW was calculated as the difference between the dry weight and the filter weight after ashing.

### Pigment Analysis

Chlorophylls a and c1+c2 were quantified using the spectrophotometric method of Ritchie (2006). 100 μL of culture was centrifuged at 8,000 g for 5 minutes at 4° C., the supernatant was aspirated, and the pellets were frozen at −20° C. Samples were placed on ice in the dark and extracted with 1 mL of 100% methanol for 20 minutes followed by centrifugation at 15,000 rpm for 10 minutes at 4° C. 0.7 mL of the extract was transferred to a cuvette and the absorbance was measured at 632 and 665 nm.

### Fatty Acid Methyl Ester (FAME) Analysis

Lipids were quantified by GC-FID FAME analysis using the methods of Work et al. (2010). 0.5 mL of culture was centrifuged at 8,000 g for 5 minutes at 4° C., the supernatant was aspirated, and the pellet was frozen at −20° C. The pellet was thawed, and the lipids were saponified by resuspension in 1 mL methanol saturated with NaOH and heated in sealed vials at 100° C. for 90 minutes. The vials were cooled to room temperature and acid-catalyzed methylation was achieved by adding 1.5 mL of methylation reagent (14.6 mL 12 N HCl+235.4 mL MeOH) and incubated at 60° C. overnight. FAMEs were extracted by adding 1.25 mL n-Hexane and gently inverting for 30 minutes on a shaker table. Extracts were analyzed by gas chromatography-flame ionization detection (GC-FID) using an Agilent 7890A gas chromatograph with a DB5-ms column (Agilent Technologies, Santa Clara, CA) and quantified by comparison with a Supelco 37 component FAME standard (Sigma-Aldrich, USA).

### Protein Extraction and Quantification by Modified Lowry

Cultures were sampled in-situ and 0.5 mL aliquots were centrifuged at 5,000 g for 5 minutes, the supernatants were aspirated, and the pellets were frozen at −80° C. for later analysis. Pellets were resuspended in lysis buffer comprised of 50 mM Tris-HCl pH 8, 75 mM NaCl, 5% glycerol, and 1% SDS. Resuspended samples were kept on ice and probe sonicated for 30 seconds in ice water and returned to ice between rounds of sonication. Three rounds of sonication were performed per sample. Sonicated samples were centrifuged at 15,000 RPM, 4° C. for 10 minutes. The supernatant was then analyzed using the Bio-Rad DC modified Lowry protein assay kit using the manufacturer's specifications.

### Carbohydrate Analysis

0.5 mL of culture was removed from the culture bottle and immediately frozen at −20° C. For each sample, 900 μL of anthrone reagent (2 g L⁻¹ anthrone, 71% 10 N H2SO4+29% H2O v/v) was aliquoted into a 1.7 mL tube and chilled in an ice water bath. 100 μL of thawed sample was added to the chilled anthrone aliquots and mixed by inversion. Once all samples and D-glucose standards (0-500 mg d-glucose L⁻¹ in H2O) were prepared, the tubes were transferred simultaneously to a boiling water bath and left for exactly 12 minutes. The tubes were then simultaneously returned to the ice water bath to stop the reaction. The tubes were vortexed, 200 μL of each sample was transferred to a 96-well plate, and the absorbance at 625 nm was measured in a Synergy 2 plate reader (Bio Tek, USA).

### Calculation of Gallons of Gasoline Equivalent (GGE)

Based on DOE guidelines, FAMEs ton⁻¹ AFDW were multiplied by 0.280 and carbohydrates ton⁻¹ AFDW were multiplied by 0.106 and summed to yield GGE ton⁻¹ AFDW. Because protein makes up a significant proportion of algal biomass, the inclusion of the industrially feasible conversion of protein to fuel, using hydrothermal liquefaction (Valdez et al. 2014) or deamination and conversion to C4 and C5 alcohols (Huo et al. 2011), was also added to the analysis with protein ton⁻¹ AFDW multiplied by 0.1.

### Example 2. Assessment of Nutrient Feeding Timing on Areal Biomass Productivity

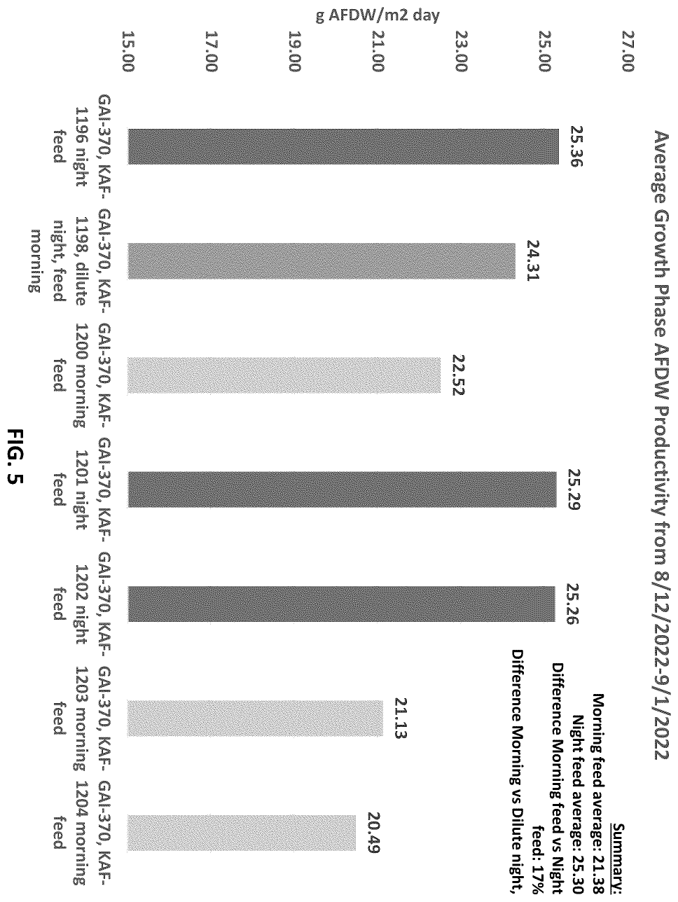

Nutrient timing and its effect on productivity was tested outdoors in ˜2m² raceways. Nitzschia inconspicua strain GAI-370, which is an oxygen tolerant strain having an improved intrinsic growth rate at a higher dissolved oxygen concentration (oxygen tolerant strains are described in U.S. Application No. 63/443,298, which is incorporated herein by reference) was cultivated outdoors for 26 days testing three different conditions as shown in FIG. 5:
1) dilution with bicarbonate media and nutrient feeding at dusk (5:00 pm) (blue bars);
2) dilution with bicarbonate media and nutrient feeding in the morning (8:00 am) (yellow bars); and
3) dilution with bicarbonate media at dusk (5:00 pm) and nutrient feeding in the morning (6:45 am) (green bar).

Conditions 1 and 2 were conducted in triplicate and condition 3 as a single replicate.

Overall, the night feeding (condition 1) outperformed the day feeding (condition 2) by 17% (p-value <0.002). Average growth productivities for all three conditions for each raceway are shown in FIG. 5. Diluting at night and feeding in the morning (condition 3) yielded a moderate increase (13%) in productivity compared to the day feeding (condition 2). These data confirm that improved productivity may be achieved by diluting the algae in the evening in outdoor conditions.

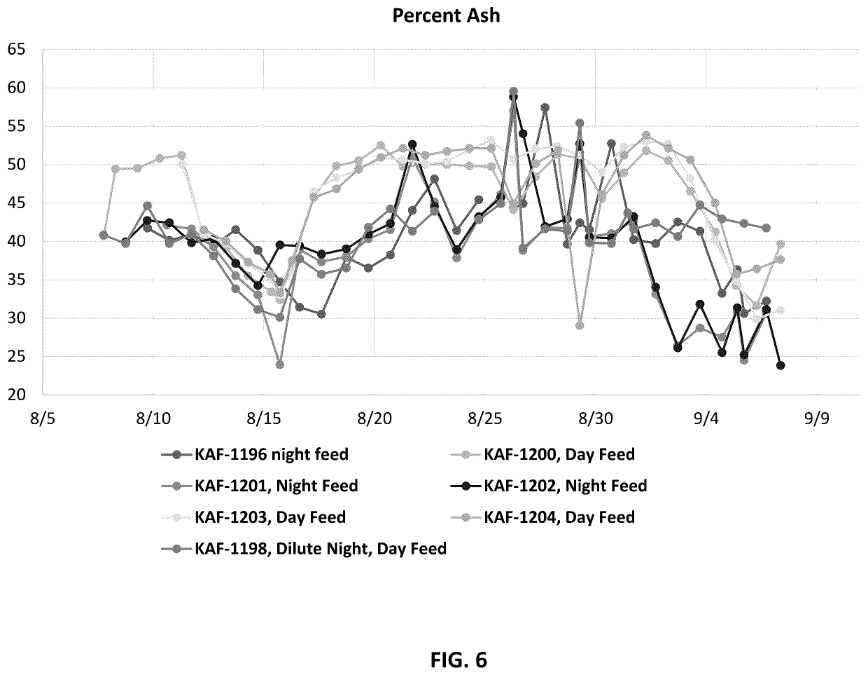

Also, the ash percentage of samples collected at dusk is lower than those collected in the morning, with averages roughly 39% and 45%, respectively (see Table 1 and FIG. 6). Lower ash content is beneficial for productivity and for rapidly forming lipids in diatoms.

TABLE 1
Average Ash Content

| | Average ash | | Average of three |
|---|---|---|---|
| Batch | content | Condition | batches |
| 1200 | 45.2 | Day | |
| 1203 | 45.5 | Day | |
| 1204 | 45.1 | Day | 45.3 |

| | | |
|---|---|---|
| 1198 | 41.3 | Dilute night, day nutrients |

| | | | |
|---|---|---|---|
| 1196 | 41.1 | Night | |
| 1201 | 38.4 | Night | |
| 1202 | 39.5 | Night | 39.7 |

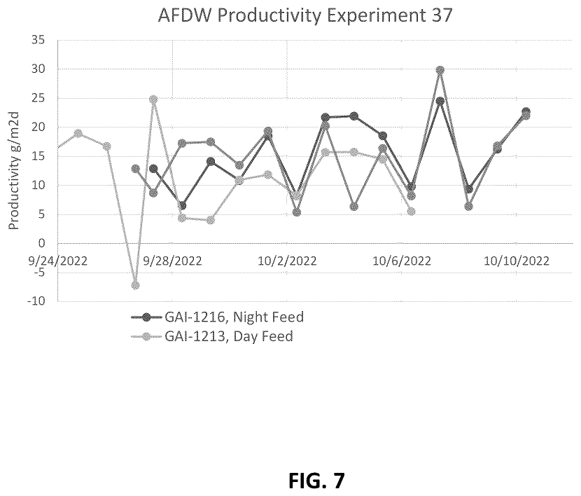

To further test if dilution or feeding was the factor in the increased productivities of night feeding, a second experiment was conducted with three conditions in single replicates as shown in FIG. 7:
1) dilution with bicarbonate media and nutrient feeding at dusk (5:00 pm) (blue line);
2) dilution with bicarbonate media and nutrient feeding in the morning (anytime between 8:30-10:30 am) (yellow line); and
3) metered dilution at night at a constant rate over 5 hours with nutrient feeding in the morning (6:45 am) (green line).

Results were similar to the previous experiment (FIG. 5), dusk feeding and dilution (condition 1) outperforming the day feeding/dilution by 42%. Metered dilution (condition 3) had a slightly lower productivity than condition 1 dusk feeding, with average productivities being 14.8 versus 15.2 g/m² day, respectively (FIG. 7). The results indicate that timing of the dilution had a greater impact on productivity than the timing of the nutrient feeding. This is likely because all nutrients were fed at the same time rather than specific nutrients at night such as luxury consumed nutrients and specific nutrients kept at constant concentration such as nitrate. Thus, there were competing effects that prevented the change in nutrient feeding from having as great an effect.

To further assess the impact of timing of the feeding on productivity, nutrient feed timing was tested during winter time and with algae cultures under direct air capture conditions. Three conditions were tested in duplicate:
1) dilution with high carbonate media and nutrient feeding at dusk (5:00 pm) with a working biomass density of 1 g/L AFDW and an average dilution rate of 21% per day;
2) dilution with high carbonate media and nutrient feeding in the morning (anytime between 8:30-10:30 am) with working biomass density of 1 g/L AFDW and an average dilution rate of 21% per day; and
3) dilution with high carbonate media and nutrient feeding at dusk (5:00 pm) with a working biomass density of 0.5 g/L AFDW and an average dilution rate of 34% per day.

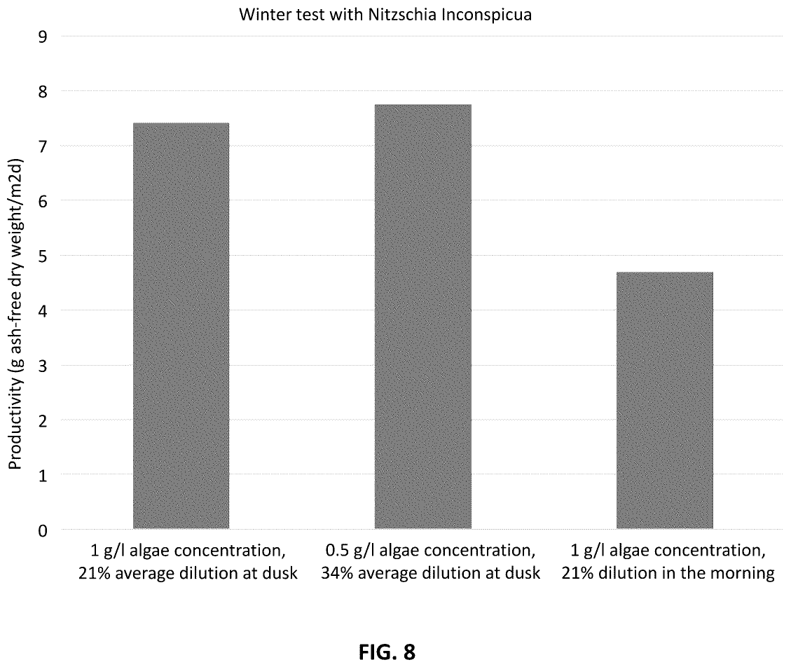

The productivity results are illustrated in FIG. 8. Again, dusk feedings outperformed the morning feedings, for this experiment by 58% for the 1 g/l AFDW, 21% dilution rate. These data, across an array of seasonal conditions, demonstrate a notable difference in nutrient feedings at dusk versus during the daytime morning hours. Data suggests that dilution timing contributes to productivity, though not as strongly as nutrient feeding timing.

### REFERENCES CITED

1. Oliver, Aaron, Sheila Podell, Pinowska Agnieszka, Jesse C Traller, Sarah R Smith, Ryan McClure, Beliaev Alex, et al. 2021. “Diploid Genomic Architecture of Nitzschia Inconspicua, an Elite Biomass Production Diatom.” Scientific Reports (Nature Publisher Group) 11 (1). https://doi.org/10.1038/s41598-021-95106-3.
2. Work, Victoria H., Randor Radakovits, Robert E. Jinkerson, Jonathan E. Meuser, Lee G. Elliott, David J. Vinyard, Lieve M. L. Laurens, G. Charles Dismukes, and Matthew C. Posewitz. 2010. “Increased Lipid Accumulation in the Chlamydomonas Reinhardtii sta7-10 Starchless Isoamylase Mutant and Increased Carbohydrate Synthesis in Complemented Strains.” Eukaryotic Cell, August. https://doi.org/10.1128/EC.00075-10.
3. Valdez, Peter J., Vincent J. Tocco, and Phillip E. Savage. 2014. “A General Kinetic Model for the Hydrothermal Liquefaction of Microalgae.” Bioresource Technology 163 (July): 123-27. https://doi.org/10.1016/j.biortech.2014.04.013.
4. Hildebrand, Mark, Aubrey K Davis, Sarah R Smith, Jesse C Traller, and Raffaela Abbriano. 2012. “The Place of Diatoms in the Biofuels Industry.” Biofuels 3 (2): 221-40. https://doi.org/10.4155/bfs.11.157.

1. A method for cultivating an algae strain in an aqueous culture, the method comprising adding a diluent to the culture daily at no earlier than one hour before sunset and no later than two hours before sunrise.

2. The method of claim 1, wherein the entire diluent is added to the culture at one time.

3. The method of claim 1, wherein before adding the diluent to the culture each day, the method further comprises transferring the culture from a first container to a second container, wherein the second container has a greater area than the first container.

4. The method of claim 1, wherein the diluent comprises one or more of aqueous media, water, and recycled media from an algae harvest.

5. A method for cultivating an algae strain in an aqueous culture, the method comprising adding a diluent to the culture daily at no earlier than two hours before sunrise and no later than one hour before sunset, wherein the diluent is added gradually.

6. The method of claim 5, wherein before adding the diluent to the culture each day, the method further comprises transferring the culture from a first container to a second container, wherein the second container has a greater area than the first container.

7. The method of claim 5, wherein the diluent comprises one or more of aqueous media, water, and recycled media from an algae harvest.

8. A method for cultivating an algae strain in an aqueous culture, the method comprising:
a) adding a first subset of nutrients to the culture daily no earlier than one hour before sunset and no later than two hours before sunrise;
b) adding a second subset of nutrients to the culture daily no earlier than 2 hours before sunrise and ending no later than sunset, wherein the one or more nutrients are added gradually; and
c) adding a third subset of nutrients to the culture daily no earlier than two hours before sunrise and no later than two hours after sunrise.

9. The method of claim 8, wherein the first subset of nutrients comprises one or more of a phosphorous source, a calcium source, a magnesium source, a sulfur source, and an iron source.

10. The method of claim 8, wherein the first subset of nutrients and the second subset of nutrients each comprise one or more of a nitrogen source, a calcium source, a magnesium source, and a potassium source; and wherein the third subset of nutrients comprises a phosphorus source.

11. The method of claim 8, wherein the first subset of nutrients comprises one or more of a calcium source, a magnesium source, and a potassium source; wherein the second subset of nutrients comprises a nitrogen source; and wherein the third subset of nutrients comprises one or more of a calcium source, a magnesium source, and a potassium source.

12. The method of claim 11, wherein the third subset of nutrients further comprises a nitrogen source.

13. The method of claim 8, further comprising adding a fourth subset of nutrients to the culture daily such that a concentration of the fourth subset of nutrients in the culture is kept substantially constant throughout the day.
